(19) United States
(12) Patent Application Publication
(10) Pub. No.: US 2007/0082013 A1
(43) Pub. Date: Apr. 12, 2007

(54) Use of botulinum toxin for producing drug for preventing or treating final phase of lung troubles

(76) Inventor: Pierre-Etienne Chabrier De Lassauniere, Paris (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/564,825

(22) PCT Filed: Jul. 16, 2004
(86) PCT No.: PCT/FR04/01870

§ 371 Date: Jun. 16, 2006

(30) Foreign Application Priority Data
Jul. 18, 2003 (FR) 0308800

(51) Int. Cl. A61K 39/08

(52) U.S. Cl. 424/239.1

(57) ABSTRACT

The invention relates to the use of botulinum toxin for producing a drug for preventing or treating the final phase of lung troubles.

A subject of the present invention is the use of botulinum toxin for the preparation of a medicament intended to prevent or treat terminal-phase pulmonary distress.

A certain number of dying patients are affected, in the few days which precede death, by extremely unpleasant audible breathing problems during the few hours or few days before their death. Said audible breathing problems, which generally consist of persistent bothersome noises, are called “death rattles”; their origin is a “terminal-phase pulmonary distress” or a “pulmonary distress in bedridden patients”; they cause great suffering to the patient and distress for their loved ones who also hear it. This pathology often appears in patients in the last stage of cancer, in particular dying patients who have a brain tumour or lung cancer, or also in patients suffering from terminal-stage neurodegenerative diseases.

The treatment currently recommended for these patients is the administration of scopolamine, a compound known for its unpleasant side effects.

A subject of the present invention is to offer an alternative solution which is much simpler and also free from side effects. Moreover, considering the moment at which the treatment according to the invention is administered, an isolated administration will be sufficient to ensure treatment.

Botulinum toxin, in particular type A botulinum toxin (Dysport® marketed by Ipsen or Botox® marketed by Allergan), has been used since the 80s in humans for the treatment of many and varied diseases/disorders. Among the diseases/disorders which can be treated with botulinum toxin, there may be mentioned, amongst others, muscular disorders (for example blepharospasm, adult or child spasticity or also torticollis), migraine, pain in general, diabetes, hyperhidrosis (or excessive perspiration), hypersalivation or even wrinkles.

According to the invention, botulinum toxin is used to eliminate the above-mentioned audible breathing problems. Preferably, taking account of the latency between the administration of the botulinum toxin and the start of its effects, the toxin will be administered as a preventive measure to patients who are known to be in the last phase of life.

The invention therefore relates to the use of botulinum toxin for the preparation of a medicament intended to prevent or to treat terminal-phase pulmonary distress, the symptoms of which are audible breathing problems associated with dying (death rattle).

Preferably, the prepared medicament is intended to be administered as a preventive measure to the patient who may suffer from terminal-phase pulmonary distress and therefore from audible breathing problems associated with dying or death rattles. This patient is in particular a patient in the terminal-phase of cancer, and in particular a patient suffering from a brain tumour or lung cancer; this patient can also be a patient suffering from a terminal-stage neurodegenerative disease.

Alternatively, the prepared medicament is intended to be administered to treat terminal- phase pulmonary distress and therefore audible breathing problems or death rattles which have already been observed in the dying patient.

The botulinum toxin used for the preparation of a medicament according to the invention is chosen from botulinum toxins of type A (including A₁, A₂and A₃), B, C (including C₁ and C₂), D, E, F and G. Preferably, it is chosen from botulinum toxins of type A, B and F. Still more preferably, it is chosen from botulinum toxins of type A and B; in particular, it is type A botulinum toxin.

Moreover, the botulinum toxin used for the preparation of a medicament according to the invention can be in the form of a complex comprising botulinum toxin or else in free form (i.e. free of any protein complexing it).

According to the invention, the prepared medicament can be a lyophilized powder comprising botulinum toxin (in which case the doctor will reconstitute the solution with water or an aqueous saline solution before injecting it into the patient) or also an injectable solution comprising said toxin.

The prepared medicament according to the invention is intended to be injected either into the parotid gland, or into the tensor veli palatini muscle of the patient experiencing terminal-phase pulmonary distress and therefore having audible breathing problems associated with dying (death rattle).

The dose of botulinum toxin according to the present invention to be provided for the treatment of the above-mentioned audible breathing problems, varies according to the age and the body weight of the subject to be treated as well as the state of the latter, and will be finally decided by the attending doctor or vet. Such a quantity determined by the attending doctor or vet is here called “therapeutically effective quantity”.

By way of example, for type A botulinum toxin, the administration dose envisaged for a medicament according to the invention is 20 to 2 000 LD₅₀ units of type A botulinum toxin per patient, preferably 50 to 1 000 LD₅₀ units of type A botulinum toxin per patient and more preferably 100 to 500 LD₅₀ units of type A botulinum toxin per patient (for example approximately 200 LD₅₀ units of type A botulinum toxin per patient). For botulinum toxins of other types, a person skilled in the art will adjust the necessary dose according to his knowledge of the relative therapeutic activity of each of these botulinum toxins in relation to the type A botulinum toxin. LD₅₀ units are commonly used by the practitioner using botulinum toxin; one LD₅₀ unit of botulinum toxin corresponding to the equivalent dose of toxin which kills 50% of a group of 18 to 20female Swiss-Webster mice weighing approximately 20 grams each.

The term “approximately” refers to an interval around the value considered. As used in the present application,“approximately X” signifies an interval of X minus 10% of X to X plus 10% of X, and preferably an interval of X minus 5% of X to X plus 5% of X.

Unless stated otherwise, all the technical and scientific terms used here have the same meaning as that usually understood by an ordinary specialist in the field to which this invention belongs. Similarly, all the publications, patent applications, all the patents and all other references mentioned here are incorporated by way of reference.

The following examples are presented to illustrate the above procedures and should in no way be considered a limit to the scope of the invention.

### EXAMPLES

### Example 1

A patient of around sixty years of age suffering from an incurable brain tumour having a life expectancy of at most one month but not on his death bed is given a preventive injection of 150 LD₅₀ units of type A botulinum toxin (Dysport®; supplier: Ipsen) into the parotid gland in order to prevent the occurrence of audible breathing problems associated with death (death rattle).

### Example 2

A patient of around sixty years of age suffering from an incurable brain tumour and on his death bed suffers from audible breathing problems associated with his state (death rattle). He is given an injection of 250 LD₅₀ units of type A botulinum toxin (Dysport®; supplier: Ipsen) into the parotid gland in order to make said audible breathing problems disappear.

### Example 3

A patient of around sixty years of age suffering from an incurable brain tumour and on his death bed suffers from audible breathing problems associated with his state. After a local anesthesia (tetracaine hydrochloride), he is given an injection of 250 LD₅₀ units of type A botulinum toxin (Dysport®; supplier: Ipsen) into the tensor veli palatini muscle in order to make audible breathing problems disappear.

1. Use of botulinum toxin for the preparation of a medicament intended to prevent or to treat death rattles.

2. Use according to claim 1, characterized in that it is intended to prevent the occurrence of death rattles.

3. Use according to claim 1, characterized in that it is intended to treat death rattles.

4. Use according to one of claims 1 to 3, characterized in that the botulinum toxin used is chosen from botulinum toxins of type A, B, C (including C₁ and C₂), D, E, F and G.

5. Use according to claim 4, characterized in that the botulinum toxin used is chosen from the botulinum toxins of type A, B and F.

6. Use according to claim 5, characterized in that the botulinum toxin used is chosen from type A and B botulinum toxins.

7. Use according to claim 6, characterized in that the botulinum toxin used is type A botulinum toxin.

8. Use according to one of claims 1 to 7, characterized in that the prepared medicament comprises a dose of 20 to 2000 LD₅₀ units of type A botulinum toxin type A botulinum toxin.

9. Use according to one of claims 1 to 8, characterized in that the prepared medicament is in the form of a lyophilized powder.

10. Use according to one of claims I to 8, characterized in that the prepared medicament is in the form of a liquid solution ready to be injected into the patient.
